(19) United States
(12) Patent Application Publication
(10) Pub. No.: US 2022/0160989 A1
(43) Pub. Date: May 26, 2022

(54) MODULAR VENTILATOR WITH VENTURI-BASED OXYGEN CONTROL

(71) Applicant: World Ventilator Foundation, Seattle, WA (US)

(72) Inventor: Ronald Tobia, Seattle, WA (US)

(73) Assignee: World Ventilator Foundation, Seattle, WA (US)

(21) Appl. No.: 17/535,306

(22) Filed: Nov. 24, 2021

Related U.S. Application Data
(60) Provisional application No. 63/118,584, filed on Nov. 25, 2020.

(51) Int. Cl. A61M 16/12; A61M 16/00; A61M 16/20

(52) U.S. Cl. CPC A61M 16/127; A61M 16/0051; A61M 2016/1025; A61M 16/204; A61M 16/205; A61M 16/209

(57) ABSTRACT

A modular ventilator according to the present disclosure may include a ventilator core and a ventilator service module. The ventilator core provides the basic functionality necessary for delivering suitably oxygenated air to a patient without most or all typical patient monitoring functions except a basic alarm or safety alert triggered by loss of pressure at the output. Additional patient monitoring functions are embodiment in the removable ventilator service module, which may be powered by its own power source and/or by the power source of the ventilator core when coupled thereto. The ventilator core is configured for low cost manufacture and ease of operation and may be portable so as to be easily deployable in a non-hospital setting. The ventilator core may employ a venture-based O₂ regulator for adjusting the oxygen-air mixture at the output, which may facilitate the manufacture of the ventilator core at lower cost than conventional ventilators.

### CROSS-REFERENCE TO RELATED APPLICATION(S)

This application claims priority to U.S. Application No. 63/118,584 filed Nov. 25, 2020, which is incorporated herein by reference, in its entirety, for any purpose.

### FIELD

The present disclosure relates generally to breathing assistance apparatus, and more specifically to a modular ventilator having a ventilation core removably coupled to a patient-monitoring service module and a venturi-based oxygen-air mixture adjustor incorporated in the ventilation core.

### BACKGROUND

Mechanical ventilation is a life-support system used to maintain adequate lung function in a patient who is unable to breathe, or is breathing insufficiently, on their own, such as a patient who is critically ill or is under anesthesia. As such a mechanical ventilator, or simply ventilator, is a machine that provides mechanical ventilation by moving breathable air into and out of the lungs of such a patient. Modern ventilators are typically complex, computerized microprocessor-controlled machines. A patient can also be ventilated with a simple, hand-operated bag valve mask, however manual ventilation is typically only used for a short period of time, such as during transport of a patient to a hospital, and are generally impractical for long term care. If a patient requires longer period of ventilation, such as in intensive-care medicine or home care, a ventilator is used. Ventilators are expensive and can be difficult to properly operate without appropriate training.

In a pandemic disease crisis, the supply of critical care ventilators needed for respiratory support of patients may be strained, which may lead to unnecessary and potentially avoidable deaths. Having a “strategic reserve” of ventilators, ideally on a global scale, which can be available for immediate use when a pandemic disease emerges, particularly in densely populated and economically poorer areas, can be desirable as demonstrated by the Covid-19 pandemic. However stockpiling expensive ventilators may not be practical, particularly for economically challenged countries or regions. It may therefore be desirable to have available a ventilator that can both meet modern ICU standards, such as standards of care for critically ill patients for in hospital use, while still being affordable and capable of easy deployment outside of a modern hospital environment, such as in poorer regions of the world, in a battle field and/or in transport environments.

### BRIEF DESCRIPTION OF THE DRAWINGS

The accompanying drawings, which are incorporated in and constitute a part of the specification, illustrate examples of the disclosure and, together with the general description given above and the detailed description given below, serve to explain the principles of these examples.

FIG. 1 is a block diagram of a modular ventilator in accordance with the present disclosure.

FIG. 2 is a pneumatic circuit of a ventilation core of a modular ventilator according to the present disclosure.

FIG. 3A is a block diagram of an electronic controller of a ventilation core of a modular ventilator according to the present disclosure.

FIG. 3B is a process flow diagram of a control process of the ventilation core.

FIG. 4 is a block diagram of an example electronic controller of a service module of a modular ventilator according to the present disclosure.

FIG. 5A-5C illustrate different states of an apparatus for regulating the oxygen concentration of the inspiratory flow.

FIGS. 6A and 6B show an isometric and cut-away view of an example implementation of a flow splitter for an oxygen concentration regulation apparatus, such as the one shown in FIGS. 5A-5C.

FIGS. 7A and 7B show a side cutaway view and a front view showing a first setting of the flow splitter in FIGS. 6A and 6B.

FIGS. 8A and 8B show a side cutaway view and a front view showing a first setting of the flow splitter in FIGS. 6A and 6B.

FIGS. 9A and 9B show a side cutaway view and a front view showing a first setting of the flow splitter in FIGS. 6A and 6B.

FIG. 10 is a schematic illustration of flow splitter for an oxygen concentration regulation apparatus according to another example of the present disclosure

FIG. 11 is a schematic illustration of flow splitter for an oxygen concentration regulation apparatus according to yet another example of the present disclosure

FIG. 12 is an illustration of a ball and spring detent that may be used for delineating discrete settings in a flow splitter according to the present disclosure.

The drawings are not necessarily to scale. In certain instances, details unnecessary for understanding the disclosure or rendering other details difficult to perceive may have been omitted. In the appended drawings, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label. The claimed subject matter is not necessarily limited to the particular examples or arrangements illustrated herein.

### DETAILED DESCRIPTION

As noted, a substantial need exists for a ventilator that can be manufactured at a lower cost, one which is easier to operate and can be easily deployed in emergency situations such as in a pandemic and/or in non-hospital settings. An example of such a low cost ventilator is the modular ventilator according to the present disclosure. The ventilator of the present disclosure has modular design whereby the essential or critical functions necessary for providing breathing support to a patient are included in an easy to use, and inexpensive to manufacture, ventilation core. The ventilation core preferably includes only those critical features that meet the needs of a patient, such as a patient in intensive care. The ventilation core is configured to be operated safely and easily by a provider that may not have the specialized training necessary for operating highly technical, modern ICU ventilators. The ventilation core is a modular unit that is separable from and can be operated independent of a service module. The service module, which may be coupled to and removed from the ventilation core, provides additional functionality that enable the modular ventilator to comply with modern hospital equipment standards, such as the ISO 80601-2-12 Critical Care Ventilator standard. The add-on service module may thus be interchangeably referred to as patient monitoring module.

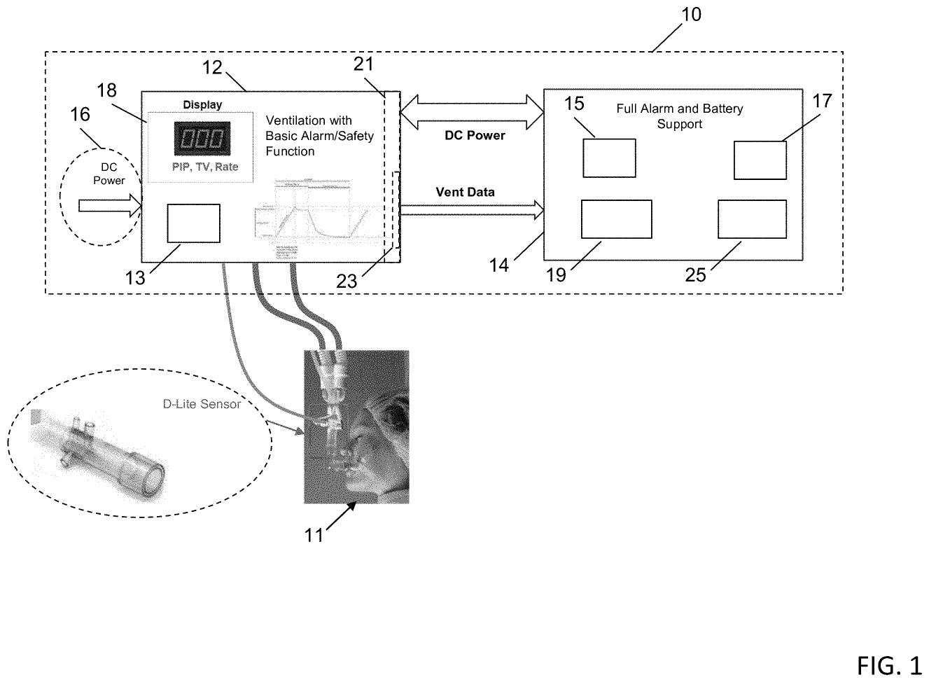

FIG. 1 shows an block diagram of a modular ventilator 10 according to the present disclosure. The modular ventilator 10 in FIG. 1 includes a ventilation (or ventilator) core 12 and a service module 14 which is removably coupled to the ventilation core 12. The ventilation core 12 may be powered by an external power source 16 (e.g., a DC power supply). In some embodiments, the ventilation core 12 may additionally or alternatively be powered by an optional internal power source (e.g., a rechargeable battery). The ventilation core 12 is a stand-alone unit that can be operated independent of the service module 14 and thus includes or is connectable to a power source irrespective of whether the service module 14 is coupled thereto. In some embodiments, the ventilation core 12 may also supply power to the service module 14 when connected thereto. The ventilation core may be equipped with one or more electrical connectors 21 for electrically coupling the service module 14 to the ventilation core 12. In some embodiments, separate connectors may be used for transmitting data from the ventilation core 12 to the service module 14 and for providing power to the service module 14. In other embodiments, a single connector (e.g., a USB port) may be used for transmitting both data and power to the service module 14. In preferred embodiments, the operation of the ventilation core 12, such as the operation of the display 18 and electronically-controlled components of the pneumatic system within the ventilation core, is controlled by an electronic controller 13, examples of which are described further below.

In preferred embodiments, the ventilation core 12 includes only those components and functions necessary to provide a preset flow rate and breath cycle timing to a patient 11 when the ventilation core 12 is connected to a source of pressurized oxygen (O₂). The ventilation core, which is operable as a stand-alone unit separate from the service module, includes bare bones functionality to provide breathing assistance, in a single-fault device, safe from barotrauma and loss of ventilation hazards. As such, the ventilation core may monitor only very basic functions associated with patient ventilation, such as the airway pressure and/or flow rate to the patient 11 and may be configured to generate a fault signal for sounding an alarm, a buzzer, or visually displaying the alarm, in the event of loss of airway pressure or insufficient tidal volume. Data associated with monitoring other functions or breathing parameters may be generated by the ventilation core but may be coupled to the communication interface 23, rather than to a display or other user-interface component of the ventilation core, such that when a service module 14 is connected to the ventilation core (e.g., via the communication interface 23), the data can be transmitted to the service module 14 for generating additional patient monitoring alarm information and providing it to a medical service provider caring for the patient 11. In some embodiments the ventilation core 12 includes only basic and inexpensive components, e.g., a venturi-based, mechanically adjustable, oxygen concentration regulator, such that the ventilation core may be inexpensive to manufacture (e.g., at costs on the order of 700 USD or less).

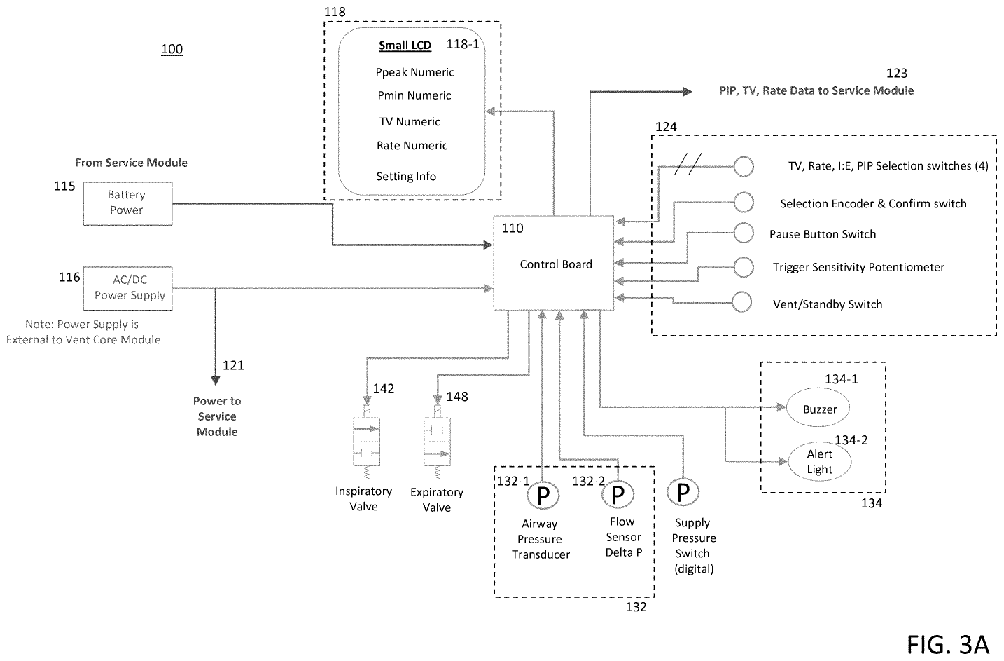

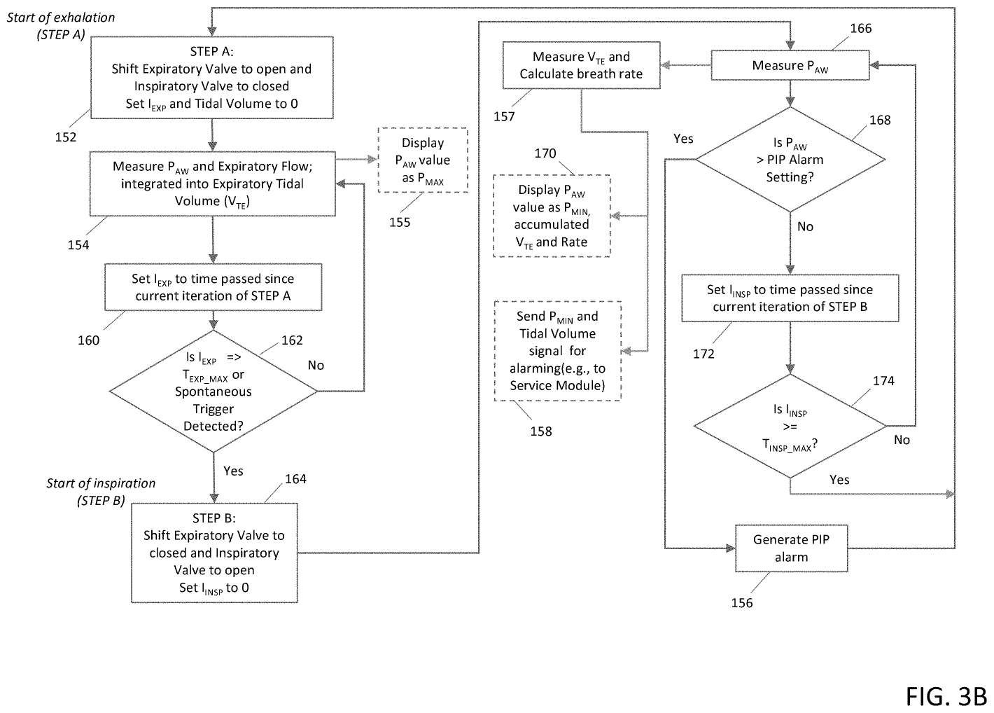

In some embodiments, the ventilation core 12 may be equipped with a display 18 (e.g., an LED or LCD display) for example for displaying critical ventilation parameters (e.g., peak inspiratory pressure (PIP), tidal volume (V_{T}), respiratory rate (RR), etc.) along with current ventilation settings. In some embodiments, an alarm or fault message generated by the ventilation core 12 may, additionally or alternatively, be provided via audio feedback, such as by sounding an audible alarm (e.g., activating a buzzer). To that end, the ventilation core may be equipped with any suitable audio output device (e.g., a speaker, a buzzer 134-1, etc.). In some embodiments, an alarm light 134-2 (see FIG. 3A) may be activated to provide an alarm generated by the ventilation core, in addition or alternatively to providing the alarm audibly and/or via the display 18. In this manner, the vent core is capable of unilaterally (i.e. without a service module) notifying users of critical alarm conditions (e.g. High airway pressure (P_{AW}), Circuit Disconnect) that have been detected by the vent core's monitoring.

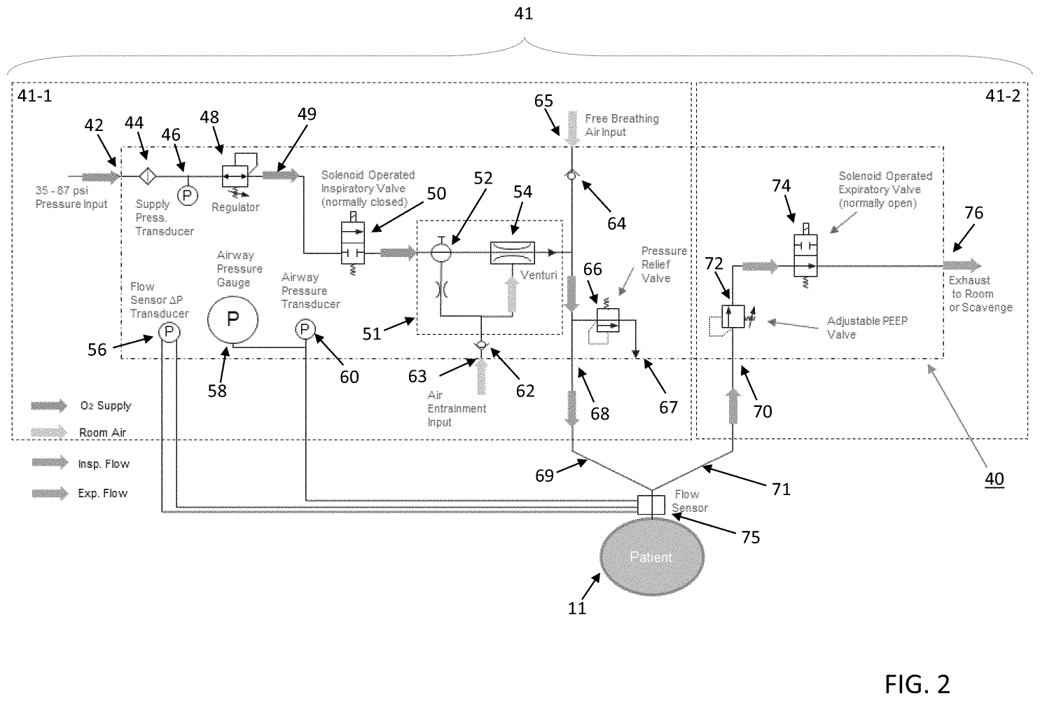

Components and operation of a ventilation core are further described with reference now also to FIG. 2, which shows a pneumatic diagram of the pneumatic subsystem 41 within a ventilation core enclosure 40 according to some examples of the present disclosure. The ventilation core enclosure 40 may be used to implement, in part, the ventilation core 12 of the modular ventilator 10 of FIG. 1. The pneumatic subsystem 41 includes an inspiratory valve 50, which is part of a first (inspiratory) portion 41-1 of the pneumatic subsystem 41 of the ventilation core 40, and an expiratory valve 74, which is part of a second (expiratory) portion 41-2 of the pneumatic subsystem 41.

The ventilation core enclosure 40 includes access to an inlet port 42 configured to be coupled to a source of pressurized oxygen. The inlet port 42 may be equipped with any suitable fitting, such as a threaded fitting, a luer fitting or other, for coupling a conduit (e.g., hose or tubing) that supplies the pressurized oxygen to the pneumatic subsystem 41 of the ventilation core enclosure 40. An optional filter 44 may be provided immediately downstream of the inlet port 42 to remove any impurities from the supply oxygen. The pressure of the supply oxygen may optionally be measured by a pressure sensor, shown here as supply pressure transducer 46, e.g., to detect a drop in pressure at the source, at the inlet port 42, as drop in the pressure of the supply gas may adversely affect the operation of the ventilation core and its ability to move sufficient amount of oxygen into the patient's lungs.

The oxygen at the inlet port 42 may be at higher pressures of variable level (e.g., in the range of 35-87 psi). Thus, the pressure of the oxygen supplied at the inlet port 42 may be reduced to a set operating pressure, which may also be referred to as the regulated pressure, by a pressure regulator 48 is just downstream of the optional filter 44 and pressure transducer 46. In this example, the pressure regulator 48 is located in the fluid path 49 upstream of the inspiratory valve 50. As such, the pressure regulator 48 is configured to provide, at its output, a flow of oxygen at the constant, regulated pressure to the inspiratory valve 50. The pressure regulator 48 may be implemented using any suitable pressure-reducing valve (e.g., a piston-based, a membrane-based, multi-stage valve, etc.), the set (or regulated) pressure for which may be set electronically or by mechanical means (e.g., varying a biasing or closing force on spring pressing on the regulating piston). In any case, the pressure at the output of the regulator 48 is the regulated pressure in this embodiment. In other embodiments, the reduction of pressure of the circuit 41 may be achieved differently.

Flow through the inspiratory limb 41-1 of the pneumatic subsystem 41 is controlled by an inspiratory valve 50 and, similarly, flow through the expiratory limb 41-2 is controlled by an expiratory valve 74. In this example, the inspiratory valve 50 is an electronically-controlled normally closed valve, which shifts to the open position responsive to a control signal. The shifting of the inspiratory valve 50 to the open position cycles the mechanical ventilator to the inspiratory phase during which air is pushed into the patient's lungs. The inspiratory valve 50 is positioned in the fluid path that delivers supply O₂ to the inspiratory port 68 to selectively control (i.e. open and close) the fluid path between the source of oxygen and then patient 11. As such, when the inspiratory valve 50 is in its normally closed position, flow through the valve 50 and thus between the inlet port 42 and the inspiratory port 68 is prevented. When the inspiratory valve 50 shifts to the open position, responsive to control signal from electronic controller (e.g., controller 100), flow of supply oxygen from the inlet port 42 to the patient, through inspiratory port 68, is permitted. The control signal may be based on any suitable variable associated with the flow, such as pressure, time, or volume. As such, the inspiratory valve 50 selectively fluidly couples the flow of supply oxygen from the inlet 42 to the downstream components (e.g., oxygen concentration regulator 51 and inspiratory port 68) of the inspiratory limb 41-1 of the circuit. In the specific example in FIG. 2, the inspiratory valve 50 is provided downstream of the pressure regulator 48 and upstream of the oxygen concentration regulator 51.

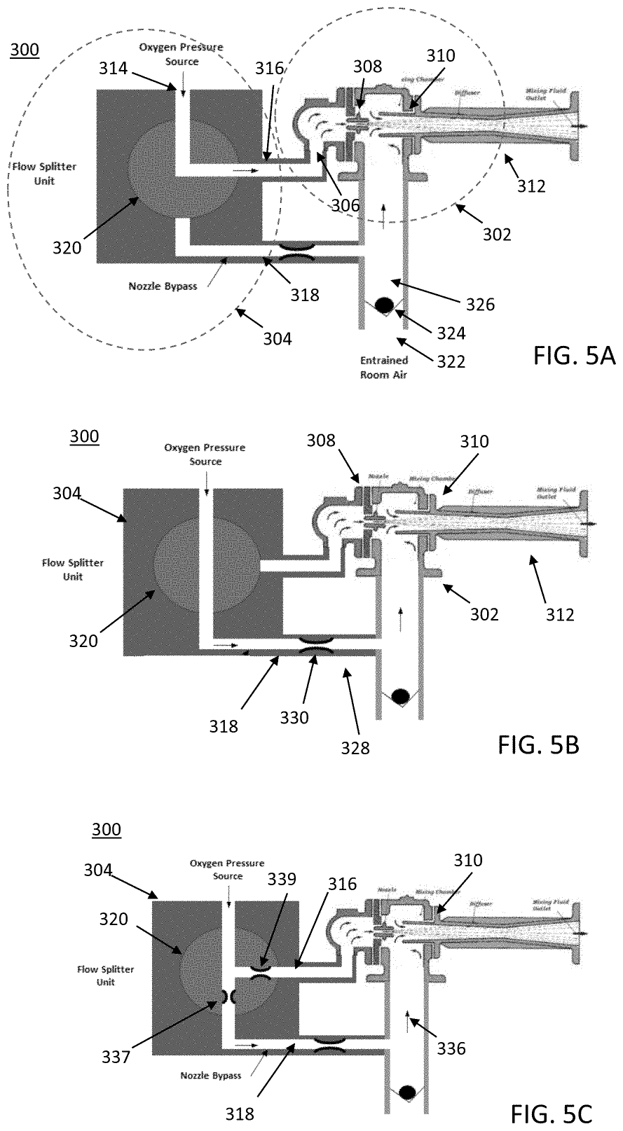

As previously noted, the pneumatic subsystem 41 includes an oxygen concentration regulator 51 configured to adjust the concentration of oxygen in the flow to provide a regulated flow of inspiratory gases, also referred to as the inspiratory flow, for delivery to the patient. In this example, the oxygen concentration regulator 51 is implemented using a venturi-based apparatus, which is easy to manufacture and operate, and examples of which are described in further details with reference to FIGS. 5A-12. An oxygen concentration regulator 51 according to the present disclosure includes a venturi device 54 configured to adjustably entrain ambient air with the flow of oxygen through the venturi's nozzle to comprise a regulated flow of inspiratory gases. The amount of air entrained by the venturi device 54 is controlled by a flow splitter (also referred to as flow diverter) 52 which enables oxygen flow to be vectored into the entrainment section of venturi device 54, reducing the amount of air entrained. Based on the setting of the flow splitter 52, the inspiratory flow, may be an oxygen-air mixture having a desired concentration of oxygen (e.g., ranging between about 60% and about 100% oxygen). The flow splitter 52 may also be configured to enable the regulator 51 to provide a consistent inspiratory flow rate (e.g., constant volumetric flow) at its output regardless of the selected oxygen concentration. That is, even if a relatively smaller amount of oxygen is passed through the flow splitter, a correspondingly larger amount of air is entrained in the flow to provide a constant flow rate at the output of the venturi-based apparatus.

In the illustrated embodiment, the inspiratory limb 41-1 includes a pressure relief valve 66 that selectively couples (upon reaching a threshold pressure) the fluid line of the inspiratory flow to a pressure relief port. The pressure relief valve 66 in this example is a normally closed valve which shifts to the open position when the pressure in the line reaches a predetermined pressure value (e.g., peak inspiratory pressure (PIP)). In this manner the pressure relief valve 66 is configured to prevent the pressure in the inspiratory flow line from exceeding the predetermined threshold (e.g., PIP). The pressure relief valve 66 may be electronically controlled, e.g., responsive to controller 13, which may be implemented by the example controller 100 in FIG. 3A. The controller may transmit a control signal to open the valve 66 based on pressure measurements obtained by the flow sensor 75 and pressure transducer 60, as described further below.

The inspiratory port 68 is configured to transmit the regulated, inspiratory flow out of the ventilation core enclosure 40 for delivery to the patient. For example, the inspiratory port may be equipped with any suitable fitting for connecting a hose or tubing 69 for delivering the inspiratory gases to the patient. The inspiratory limb 41-1 of the present example includes two additional ports open to ambient (or room) air. The first ambient air port 63 couples ambient air to the entrainment line of the venturi-based regulator 51. The second ambient port 65 provides a free-breathing air input into the ventilation core's pneumatic subsystem 41. First and second check valves 62 and 64, respectively, are associated with respective one of the first and second ambient air ports 63 and 65, respectively, to prevent outflow of gas through these ambient air ports such that pressure can be maintained in the pneumatic subsystem 41 (e.g., in the inspiratory limb 41-1). As such, the ports 63 and 65 are one-way ports allowing air to enter the pneumatic subsystem 41 but preventing air from exiting the circuit through these ports.

The flow of gas through the second (expiratory) portion or limb 41-2 of pneumatic subsystem 41 is controlled by expiratory valve 74. The expiratory valve 74 selectively permits or prevents gas from flowing from the expiratory port 70 to the ventilator scavenging outlet 76. The outlet 76 is configured to exhaust the air received from the subject, through expiratory port 70, out of the ventilation core. The expiratory valve 74 in the present example is an electronically-controlled normally open valve which shifts, responsive to a control signal from controller (e.g., controller 100), to the closed position to cycle the pneumatic subsystem 41 to the inspiratory phase. The inspiratory and expiratory valves 50 and 74, respectively, are operatively connected (e.g., via the controller) to operate in synchrony. That is, the inspiratory valve 50, which is a normally closed valve, opens when the expiratory valve 74, which is a normally open valve, closes and vice versa. An adjustable flow control device or PEEP valve 72 may be provided between the expiratory port 70 and the expiratory valve 74 to maintain pressure in the fluid line to a set pressure (e.g., a set positive end expiratory pressure (PEEP) value) to ensure that pressure in the expiratory limb 41-2 does not fall below the set PEEP pressure when the expiratory valve 74 is opened. The PEEP valve 72 allows expiratory flow to proceed to the expiratory valve 74 when expiratory port pressure is higher than the set PEEP pressure level, but shifts to a closed position, shutting off the flow of air through the expiratory limb when the expiratory port pressure is equal or below the set PEEP pressure level. The PEEP valve 72 may be mechanically, electrically or pneumatically operated to achieve the PEEP pressure control. In an alternate embodiment of the present invention, the PEEP pressure regulating function can be integrated within expiratory valve 74 using proportional electrical control (e.g. valve closure force is controlled with a proportional voice coil).

In use, a Y-piece is typically used to connect the ventilation core (e.g., to the enclosure 40), and more specifically the patient ends of both the inspiration and expiration tubes 69 and 71, respectively, to the tracheal intubation tube of the patient 11 thereby forming a machine-patient breathing circuit therewith. A flow sensor 75, such as a the D-LITE spirometry sensor sold by GE HEALTHCARE, may be provided within the machine-patient circuit, typically as close to the patient 11 as possible, e.g., between the y-piece and the intubation tube, to monitor airway pressure (P_{AW}) and other parameters associated with the patient's breathing mechanics. The flow sensor 75 may be implemented by a generally tubular structure that include a flow restrictor and a plurality of ports that can be connected, via tubing, to pressure and/or pressure differential measurement components of the ventilation core (e.g., to the enclosure 40). The sensor 75 may include a pair of pressure ports typically located on opposite sides (upstream and downstream) of the flow restrictor. The pair of pressure ports may be connected to a pressure transducer (e.g., pressure transducer 56) to measure a pressure differential across the restriction. The pressure at the restriction may also be measured, via an additional port connected to another pressure transducer 60. The pressure at the restriction and/or the pressure differential measurements obtained by transducers 56 and 60 may be used (e.g., by the controller 100 of the ventilation core and/or by a service module 14) to determine various airway flow and airway pressure parameters. Any suitable flow sensor 75 capable of monitoring at minimum the airway pressure (P_{AW}) may be used in the machine-patient breathing circuit. In some embodiments, the flow sensor 75 facilitates measuring additional flow parameters beyond the basic and minimum measurement(s) needed for patient safety (e.g., the airway pressure measurement obtained by transducer 60), which can be used by a service module when connected to the ventilation core, to provide additional patient monitoring functions.

In some embodiments, the airway pressure measured by transducer 60 is coupled to a controller, which may sound an alarm and/or visually display an alarm message, if the airway pressure reaches a certain maximum threshold (e.g., a peak inspiratory pressure (PIP)). A visual indication of the airway pressure may optionally be provided, e.g., directly and in real-time, such as via a pressure gauge 58 connected to the transducer and/or via the controller on a display (e.g., display 18 in FIG. 1 or 118 in FIG. 3A). Other alarms and/or messages that may be generated may include an alarm indicating loss of pressure in the circuit 41, a message displaying the tidal volume (TV) as may be computed based on flow parameters sensed by flow sensor 75, an alarm indicating low battery status, etc.

As previously noted, the ventilation core (e.g., ventilation core 12 of FIG. 1) may include an electronic controller 13, which may be implemented using any suitable processor or microprocessor. In an embodiment of the ventilation core, an inexpensive microprocessor, such as an Arduino or a Teensy Control Board, was used to operate the ventilation core. FIG. 3A shows a block diagram of an example controller 100 (e.g., control board 110) of a ventilation core and associated signals from components communicating with the controller. In some embodiments, the controller 100 may be implemented by a control board 110 (e.g., a control board provided by ARDUINO or TEENSY), however any suitable combination of processing and memory units may be used (e.g., a micro controller) to implement the controller 100. In the example controller 100 in FIG. 3A, the control board 110 is configured to receive various inputs and provide various outputs. The control board 110 may be powered by external power source 116 and/or an internal power source 115. The internal power source 115 may include one or more rechargeable batteries of any suitable technology (e.g., Li-ion battery). In some embodiments, e.g., in which the ventilation core is connectable to external power 116, the ventilation core may also provide a power signal 121 (e.g., from the external power source 116) to a service module 12 when connected to the ventilation core (e.g., when switch 200 is closed). That is, power from an external power source 116 may pass through the ventilation core to the service module, which may be connected thereto by any suitable connection such as a standard USB connection. In some embodiments of the ventilation core, such as ones that only include an internal power source, the internal power source 115 of the ventilation core may be supplied by the service module, via a suitable connection (e.g., a USB or other) communicatively coupling the two.

A key function of the controller 100 is to control the opening and closing of the inspiratory and expiratory valves 50 and 74, respectively. The controller 100 may send control signals 142 and 148 to the inspiratory and expiratory valves 50 and 74, respectively, for controlling the opening and closing of the valves. The valve control signals 142 and 148 for controlling the operation of the inspiratory and expiratory valves 50 and 75, respectively, are generated responsive to one or more signals 124 corresponding to parameter settings and/or measurement signals (e.g., pressure measurements 132). For example, the closing and opening of the valves may occur at set time intervals, and the timing may be set by the user (e.g., via an encoder or other user control). In some embodiments, an expiratory time encoder may be used to set the timing parameter for the closing and re-opening of the expiratory valve, for example by programming or setting a time interval for closing the expiratory valve and a duration of holding the expiratory valve in the closed position. As described, since the expiratory and inspiratory valves are configured to work in reverse synchrony, the closing of the expiratory valve may cause the opening of the inspiratory valve, and vice versa, thereby cycling the ventilation core from the exhalation phase to the inspiration phase of the breathing cycle, and vice versa. In other embodiments, the timing may be controlled differently such as by setting the time intervals for opening the inspiratory valve and a duration of holding the inspiratory valve open at each open interval, which may consequently drive the reverse synchronous operation of the expiratory valve.

The timing for opening and closing of the valves may alternatively or additionally be determined based on parameter settings and/or measurements. For example, the controller 100 may receive signals 124 from one or more switches, encoders, potentiometers or other controls that correspond to various parameter settings such as TV, breathing rate, Inspiratory time:Expiratory time (I:E) ratio, Peak Inspired Pressure (PIP), and trigger sensitivity setting that controls the synchronization of the ventilation core with spontaneous breathing of the patient, and others. The signals 124 may also include other types of control signals such as selection signals (e.g., to select and/or confirm controlling parameters or settings), pause, on/off (or vent/standby) control signals, etc. The various parameters (e.g., TV, breathing rate, I:E ratio, and PIP, trigger sensitivity, etc.) may be set via any suitable user controls such as switches, buttons, knobs, potentiometers, encoders, etc.

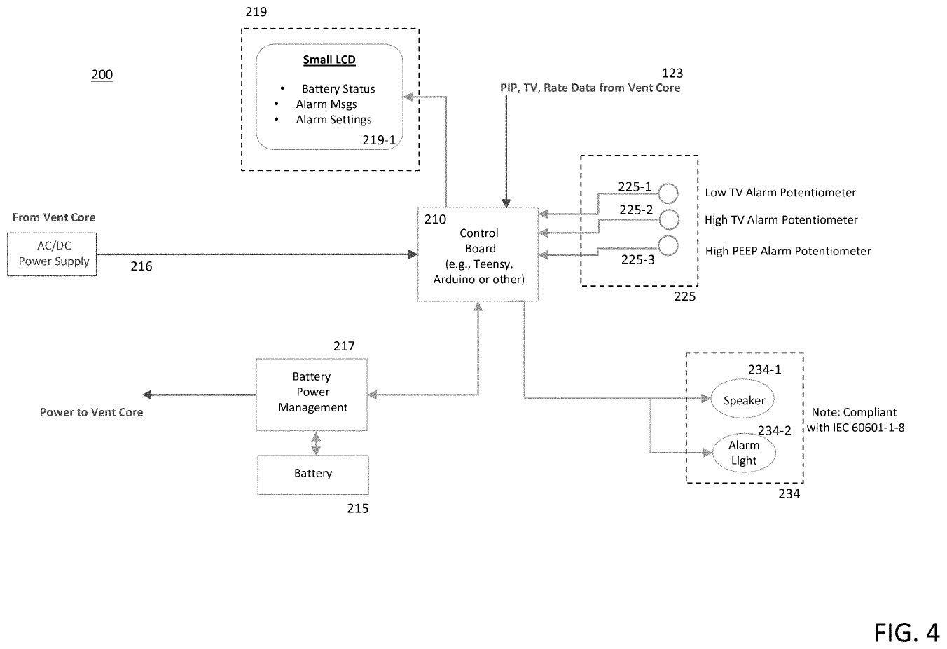

The controller 100 (e.g., control board 110) may also receive one or more pressure measurements 132, such as an airway pressure measurement (P_{AW}) 132-1 and/or a flow sensor delta P measurement 132-2 (e.g., indicative of the pressure differential across a restriction in the flow sensor 75 in FIG. 2), and which may be used to generate outputs (e.g., for display or triggering an alarm 134) and/or control the operation of one or more components (e.g., pressure relief valve 66 of FIG. 2) of the ventilation core. One or more alarms 134 may be provided audibly, e.g., via buzzer 134-1 and/or a speaker, and alternatively or optionally via an alarm light 134-2. The controller 100 may generate various output signals 118 and 123. The controller 100 may generate output signals 123 that are communicated to a service module (e.g., to controller 210 in FIG. 4) when the service module is coupled to the ventilation core. The controller 100 may generate output signals for controlling one or more displays 118, which may be implemented using any suitable display technology, showing as an example an LCD display 118-1 which is configured to display various measured parameters, alarms, status information (e.g., battery status), settings, and/or other information (e.g., error messages). While FIG. 3A shows an LCD display 118-1 as an example for implementing the display 118, it will be understood that the display 118 may be implemented using, additionally or alternatively, other display means such as multiple individual displays for individual parameters (e.g., LCD or LED displays). In some embodiments the number and/or configuration of displays may be minimized and simplified for cost considerations, and/or to facilitate a compact/portable form factor of the ventilator core. In some examples, a single display may be provided, which may be configured to display a single type of parameter (e.g., the PIP encoded into the ventilator) or may be configured to display multiple parameters (e.g., may cycle through each of a plurality of parameters such as PIP, TV, rate, measured in real-time).

FIG. 3B shows a process flow diagram of an example process 150 for controlling the inspiratory and expiratory phases of the ventilation core by a controller, described here, as an example, with reference to controller 100. The flow diagram in FIG. 3B is provided only as one example of a process for controlling certain operations of the ventilation core but it will be understood that its operation is not limited to the example in FIG. 3B. At the start of process 150, certain variables are initialized. The inspiratory and expiratory interval variables (I_{INSP}) and (I_{EXP}) that correspond to the duration of the respective time interval, are both set to 0. Maximum values of the inspiratory interval duration (T_{INSP_MAX}) and the expiratory interval duration (T_{EXP_MAX}) may be computed based on the time necessary to deliver a set tidal volume at a set breath rate. In some examples, an inspiratory to expiratory interval (I:E) ratio may be pre-set or input by the user. In some embodiments, a breathing rate (e.g., breaths per minute) and TV may be, additionally or alternatively, input by the user. In yet other examples, both T_{INSP_MAX} and T_{EXP_MAX} values may be set by the user. The PEEP value may be received via a user input or it may be pre-programmed into controller 100. The PIP may be similarly received via a user input (e.g., via a user control that generates a control signal 124 communicated to the control board 110. Some of the parameters (e.g., PEEP) that are preprogrammed and which may not be otherwise user-configurable on the ventilation core itself, may be user-configurable when the ventilation core is coupled to a service module.

As shown in FIG. 3B, the controller 100 controls the shifting of the ventilation core between the expiratory and inspiratory phases. As the ventilation core shifts from the expiratory phase to the inspiratory phase, air flow through the expiratory limb 41-2 is substantially blocked by the closing of the expiratory valve 74 while flow through the inspiratory limb 41-1 is permitted by virtue of the inspiratory valve 50 being held in the open position. Conversely, when the ventilation core shifts from the inspiratory phase to the expiratory phase, air flow through the inspiratory limb 41-1 is substantially blocked by returning the inspiratory valve 50 to its closed state while flow through the expiratory limb 41-2 is permitted by virtue of the expiratory valve 74 returning to the open state. As shown in FIG. 3B, at the start of expiratory phase (exhalation), the expiratory valve 74 is provided to an open state and the inspiratory valve 50 is provided to a closed state, as shown in block 152 (also referred to as STEP A for simplicity of illustration). The valves 74 and 50 are maintained in those states for a set period of time, specifically for the duration of the expiratory period (until T_{EXP_MAX} is reached) unless spontaneous breathing trigger is detected (as shown in block 162). During this time the controller 100 monitors the airway pressure and expiratory flow as shown in block 154. As shown in FIG. 3A, the controller 100 may receive, continuously or periodically, pressure measurements 132-1 and the 132-2. The airway pressure (P_{AW}), which may be determined by flow sensor 75 and pressure transducer 60, may optionally be displayed in real-time (e.g., via pressure gage 58 and/or on a display 118), as shown in block 155. As time passes, the expiratory interval variable (I_{EXP}) is incremented up, e.g., as shown in block 160, where the expiratory interval variable (I_{EXP}) is updated to the time that has passed since the start of the current expiratory phase iteration. As shown at decision block 162, when the expiratory interval duration (I_{EXP}) reaches the maximum set value (T_{EXP_MAX}) or if spontaneous breathing is detected, the controller 100 shifts the ventilation core to the inspiratory phase (STEP B) at block 164, otherwise the process returns to block 154 and the controller 100 continues to monitor the airway pressure and the expiratory flow and optionally display breathing parameters (at optional block 155).

At the end of the expiratory phase and start of inspiration, the controller 100 shifts the expiratory valve 74 to a closed state and the inspiratory valve 50 shifts to an open state to start the inspiratory phase, as shown in block 164. The inspiratory interval value (I_{INSP}) is reset to 0. As shown in block 166, the controller 100 continues to monitor the airway pressure (P_{AW}) during the inspiratory phase, and optionally displaying the same, as shown in block 170. In some embodiments, if the ventilation core is equipped with a display for displaying airway pressure, the airway pressure is displayed, for example in real time, irrespective of whether the ventilation core is in expiratory or inspiratory phase. As previously shown in block 155, specific sampled values of P_{AW} may be display as P_{MAX} from the previous inspiration. Similarly specific sampled values of P_{AW} may be displayed as P_{MIN} from the previous exhalation, as shown in block 170. Stated differently, block 155 captures the Pmax of the previous inspiration and block 170 captures the Pmin of the previous exhalation. Both measurements are associated with the transition (i.e. leaving exhalation or leaving inspiration) and are single value measurements for each breath. In some embodiments, the airway pressure may be displayed as a numerical value. Alternatively or additionally, the airway pressure may be displayed in the form of a graph (e.g., as a function of time), which may be generated in real time as the airway pressure is being monitored/measured. Additionally, V_{TE} is measured and breathing rate is calculated, at block 157, and the accumulated V_{TE} and breathing rate may be optionally displayed (block 170). The P_{MIN} and Tidal Volume values may be used to determine whether an alarm should be generated by the service module (block 158). The measured airway pressure is compared to the PIP value, as shown in block 168 and a PIP alarm is generated if P_{AW} is greater than the PIP alarm setting (block 156). Otherwise, the I_{INSP} is incremented up, as shown in block 172 and compared to the set maximum duration for the inspiratory interval (block 174). When I_{INSP} reaches the set maximum duration for the inspiratory interval, the inspiratory phase ends and the process returns to STEP A, shifting the ventilation core to the exhalation phase.

Prior art ventilator designs may incorporate features such as monitoring functions, alarm functions, alarm annunciation functions, battery backup etc. that enable compliance with international standards such as ISO 80601-2-12 and are necessary to gain worldwide regulatory approvals (e.g. FDA clearance). As previously noted, the current invention describes a low cost, ventilation core that is capable of providing a basic level of safe ventilation therapy to a patients in resource strained environments (e.g. pandemic crisis, third world use). For cost and complexity reasons, the ventilation core alone is not equipped to fully meet all international standards such as ISO 80601-2-12. However, the modular design of the current invention allows for attachment of an ancillary “service module” that complements and communicates with the ventilation core in order to provide additional functionality. In combination, the ventilation core and service modules are designed to meet all necessary international standards and obtain full worldwide regulatory approvals. Referring back to FIG. 1, the service module 14 is configured to provide additional patient monitoring capabilities to the module ventilator 10. The service module 14 may provide patient monitoring functions, which, when connected to the basic ventilation core 12, bring the modular ventilator 10 into compliance with additional national or world-wide standards (e.g., the ISO 80601-2-12 standard, or other FDA, CE, etc. regulatory standards for a medical device of this type). In some embodiments, the ventilation core may be configured to be compliant with certain basic safety standards such as the FDA standard for emergency ventilator, or the equivalents in other countries, while the modular ventilator 10 with the add-on service module may be compliant with a more rigorous standard such as the ISO 80601-2-12 standard applicable to medical equipment intended to be attended by a professional operator. As previously described, the service module 14 may, in some embodiments, be powered by/through the ventilation core. In other embodiments, the optional service module may have its own power source (external and/or internal). In such embodiments, the service module may also be configured to optionally power the ventilation core, for example in instances where the ventilation core is a portable unit provided only with an onboard (internal) battery. Typically, though, the ventilation core would be capable of independently being powered for normal use (e.g., to provide ventilation therapy to a patient) by its own power source, whether internal or external.

FIG. 4 shows a function block diagram of a controller 200 for a service module, such as service module 14 in FIG. 1. The service module and the controller 200 may be powered by an external power source 216 (e.g., an AC/DC power source), which may be independent of the ventilation core or which may be the power signal 121 received from the ventilation core. In some embodiments, the service module may provide power to the ventilation core. In such examples, power received from an external source 216 may be directed, via controller 200, to a power management circuit 217, which controls storage of the power in an internal battery, usage of the power by the service module, and/or delivery of power to the ventilation core. In other examples, power may be received rather than supplied to the ventilation core, and the power signals may pass through the power management circuit 217 which may direct some of the power to components of the service module such as the controller 100, display of the service module, etc., and may direct the remaining power to storage (e.g., for recharging the onboard battery 215).

The service module may be configured to provide most or all of the patient monitoring functions available on current/modern ventilators. For example, the service module may receive output signals 123 from the ventilation core controller (e.g., controller 100), which may represent various flow measurements and/or calculated breathing mechanics parameters such as PIP, TV, breathing rate, etc. Example additional monitoring functions that may be stripped from the ventilation core but instead provided by the add-on service module, may include TV monitoring, PEEP monitoring, battery status monitoring, etc. The controller 200 may receive and operate responsive to control signals 225, which may be generated responsive to one or more user controls for setting various parameters (e.g., low TV, high TV, high PEEP values, etc.). The user control(s) may be implemented using any suitable devices including one or more switches or buttons, encoders, potentiometers, or the like. In the example in FIG. 4, the control board 210 receives a first signal 225-1 which controls or sets the Low TV (or TV min) Alarm setting, a second signal 225-2 which controls or sets the High TV Alarm setting, and a third signal 225-3 that sets the High PEEP Alarm setting. In operation, one or more alarm signals 234 may be generated based on the settings associated with the first, second and third signals 225-1, 225-2, and 225-3, such as generating a low TV alarm, for example, audibly by speaker signal 234-1, visually via alarm light signal 234-2, or both if the TV drops to or below the TV min setting. Similarly a PEEP alarm may be generated by the service module, e.g., audibly by speaker signal 234-1, visually via alarm light signal 234-2, or both if the measured PEEP exceeds the high PEEP setting.

Alarms and/or other messages may be output on a display 219 of the service module, optionally additionally to alarms being output via audible or visual means (e.g., via an alarm light) that are designed to be compliant with international alarm standards (e.g. IEC 60601-1-8). The user interface of the service module may include, in addition to user controls for setting control signals 225, one or more displays 219, which may be implemented using any suitable display technology. For example, the one or more displays 219 of the service module may include an LCD display 219-1 configured to display various information such as, but not limited to, battery status, alarm settings, alarm messages, and other information such as displaying measured or calculated parameters and graphs thereof.

FIGS. 5A-5C illustrate the operation of an example apparatus 300, also referred to herein as an oxygen-air mixture adjustor or oxygen concentration regulator, and which is configured to control the concentration of oxygen in the inspiratory flow delivered to the patient. The apparatus 300 includes a venturi device 302 and a flow splitter 304.

The venturi device 302 includes a constricted section (e.g., nozzle 308), which operates to increase the velocity of the fluid (e.g., the oxygen flowing into the device 302 through inlet 306), thereby reducing the fluid's pressure, creating partial vacuum immediately downstream of the nozzle. The partial vacuum allows a second fluid, in this case ambient air, to be suctioned into and entrained with the main flow (i.e. the motive fluid) passing through the venturi device 302 to produce an oxygen-air mixture. The oxygen-air mixture is output from the venturi device 302 through an outlet 310 thereof, the outlet 310 in this example including a diffuser 312, which may allow for further mixing and pressure reduction of the two fluids of the oxygen-air mixture.

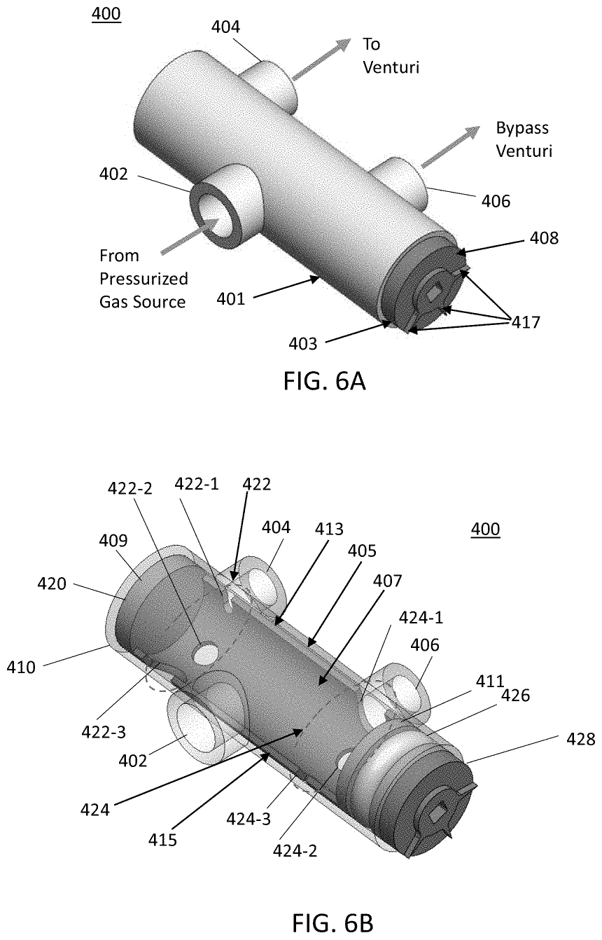

The flow of motive fluid, here oxygen, through the venturi device 302 is controlled by a flow splitter 304, exemplary implementations of which are described further below with reference to FIGS. 6-13. The flow splitter 304 includes an inlet 314, a first outlet 316 and a second outlet 318. The first outlet 316 is fluidly connected to the inlet 306 of the venturi device 302. The second outlet 318 is not fluidly connected to the venturi device 302, and may thus be referred to as venturi bypass outlet 318 or simply bypass outlet 318. A flow diverter (or simply diverter) 320 controls the amount of the flow entering the flow splitter that is coupled to the first outlet 316 and/or the second outlet 318.

In FIG. 5A, the he flow splitter 304 is placed in a state in which the oxygen pressure source is feeding the venturi nozzle 308 with substantially no flow going through the nozzle bypass. In this state, the diverter 320 of flow splitter 304 is configured or positioned such that substantially all of the flow of oxygen is coupled to the first outlet 316 and thus to the venturi nozzle 308, resulting in entrainment of ambient air and an oxygen-air mixture with reduced concentration of oxygen as compared to the flow upstream of the venturi device 302 (i.e., the concentration of the supply gas). In this state, the energy of the nozzle's flow entrains ambient (or room) air into the mixing chamber, creating an oxygen-air mixture of a first concentration. Depending on the design of the venturi device (e.g., the venturi nozzle and diffuser designs) along with the pressure source level, various mixtures and total mixed flow levels can be achieved. In one embodiment, a 50-50 mixture of Oxygen and Air is produced, which may result in an O₂ percentage of total mixed gas of approximately 60% with a total flow rate of about 40 lpm when supplied with oxygen at 25 psig.

As shown, a one-way valve (i.e. a check valve) 324 is placed in the entrainment fluid line 326 to allow air to be pulled or suctioned from ambience into the ventilation core but prevent gas from exhausting through the ambient air port 322 during exhalation periods. The check valve 324 thus prevents patient rebreathing from the inspiratory limb or portion of the pneumatic circuit and allows Positive End Expiratory Pressure (PEEP) to be maintained.

In some cases of patient care, it may be desirable to operate the ventilator in a manner such that the patient receives near or substantially 100% oxygen. In such cases, the flow splitter 304 can be provided in a full bypass state as shown in FIG. 5B. In this state, no flow is passing through the venturi nozzle 308 and consequently no air entrainment is occurring. Instead, the oxygen flows through the venturi bypass 328 and, optionally, out through the diffuser 312, whereby the inspiratory flow has substantially the same oxygen concentration (e.g., 100% oxygen) as the supply source. To that end, as shown in FIG. 5B, the diverter 320 is provided in a second configuration or position in which all of the supply flow is coupled to the second (bypass) outlet 318. In this example, since substantially none of the supply flow passes through the nozzle 308 of the venturi device, no venturi effect is produced and no air entrainment occurs. As shown, the supply flow bypasses the venturi nozzle 308 and is coupled, instead, to the outlet 310 of the venturi device 302, or just downstream thereof, whereby the flow is then provided as inspiratory flow to the patient.

As described, the ventilator core may use timed openings of its inspiratory valve in combination with a fixed inspiratory flow rate to establish a set tidal volume delivery. Thus, in such configuration, a substantially constant total flow (e.g., at 40 lpm) may need to be maintained to ensure volume delivery accuracy irrespective of the setting of the flow diverter. To that end, a pneumatic resistance (e.g., flow restrictor 330) may be placed in the bypass line to provide consistent flow rate or volume (e.g., 40 lpm inspiratory flow rate at the same 25 psig supply level) through the apparatus 300 irrespective of whether the flow passes through the venturi or bypasses the venturi.

It may be further desired from a clinical perspective to operate with mixed gases that produce an oxygen level between 60% and 100%. To achieve intermediate levels of O₂%, the flow splitter 304 can be placed in the state shown in FIG. 5C. In this configuration, the diverter 320 is provided in a configuration or position in which a portion of the supply flow is coupled to the first (nozzle) outlet 316 and the remaining portion of the flow is coupled to the second (bypass) outlet 318. The state of the flow in FIG. 5C can be described as a hybrid of the first states shown in FIG. 5A (full nozzle flow) and FIG. 5B (full bypass flow). In the configuration of FIG. 5C, an oxygen concentration different from the reduced oxygen concentration produced from the full venturi nozzle flow stat of FIG. 5A can be obtained because the entrainment fluid 336, which was previously (in FIG. 5A) substantially only ambient air, now also includes pure oxygen. The diversion of a portion of the supply flow to the bypass outlet can be achieved using one or more pneumatic resistors (e.g., first restrictor 337, second restrictor 339) upstream of the first and/or second outlets, whereby the flow through the first outlet (nozzle) outlet and the second (bypass) outlet are set to provide a smaller amount of entrained air, while still providing a desired total mixed flow rate (e.g., total mixed flow rate of 40 lpm). As such, the O₂ concentration in the fluid mixture exiting the outlet 310 is between the concentration (e.g., 60%) achieved by the first state (in FIG. 5A) and the concentration (e.g., 100%) achieved by the second state (in FIG. 5B). By varying the relative pneumatic resistances of the nozzle and venturi bypass, any number of different O₂ levels between the first (or minimum) oxygen concentration, which may be about 60%) and the second (or maximum) oxygen concentration, which may be 100% or near 100%, can be obtained without significant impact on the desired total flow rate (e.g., 40 lpm) through the ventilation core. It is further noted that a near 100% O₂ concentration may be achieved using the second state of 5C with little or no additional pneumatic resistance applied by the second restrictor 339. That is, near 100% O₂ concentrations at a constant inspiratory flow rate may be achieved with minimal change in the flow proceeding through nozzle outlet 316 and nozzle 308.

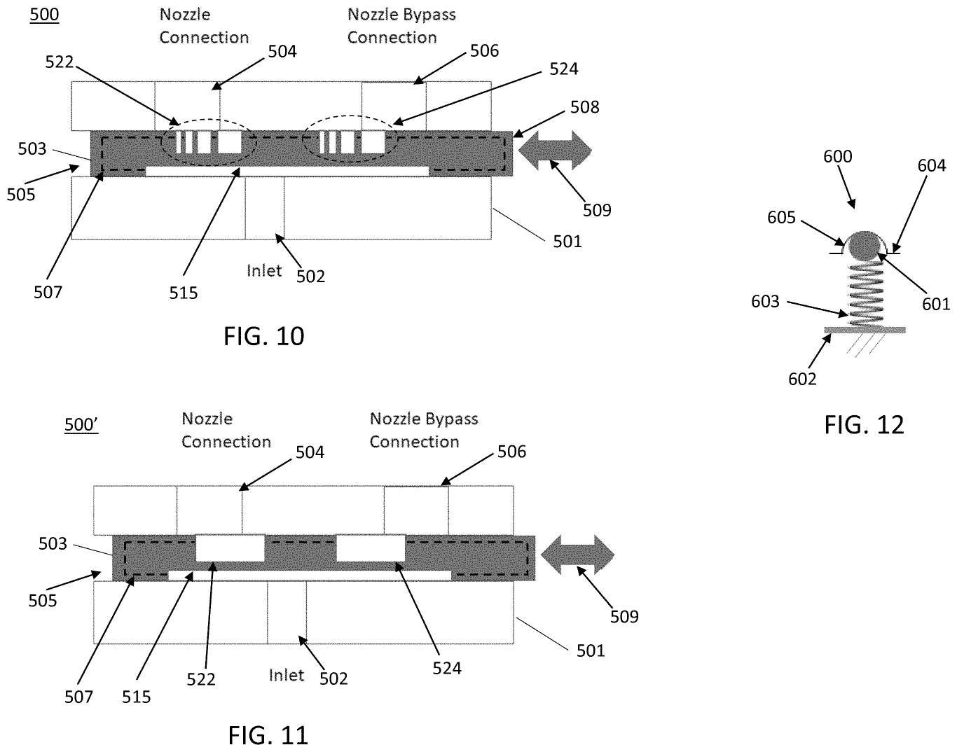

In some embodiments, a flow splitter may include a movable flow control member (or diverter) that includes a series of holes that form the pneumatic resistances of the flow splitter described in FIGS. 5A-5C connecting the high pressure inlet with connections to the venturi nozzle and the nozzle bypass. The configuration of the holes (e.g., size, relative positioning, etc.) is selected to provide desired oxygen concentrations at the output of the venturi device while maintaining a constant inspiratory flow. In some embodiments, the pneumatic resitance provided by the flow control member is adjustable by rotation of the flow control member. In one setting, a first series of holes are aligned with the outlets connected to the enturi nozzle and the nozzle bypass, with the larger hole and thus greater portion of the flow feeding the nozzle bypass, producing less entrainment, to provide an O₂% mixture of approximately 90%. When the diverter is rotated counter-clockwise, a second series of holes become engaged with the outlet connections. These holes are more equal in size and accordingly, allow more flow to go to the venturi nozzle and less through the bypass, thereby creating more air entrainment and a lower O₂% in the output mixture. The diverter would also be equipped with positions (not shown) where all flow is going through the nozzle (i.e. bypass connection is occluded without a hole) and similarly, where all flow is going through the bypass (i.e. nozzle connection is occluded without a hole). In this manner, the edge states of 60% and 100% O₂ mixture levels are implemented. As previously noted, in one embodiment the size of holes selected in the diverter would result in a total flow of 40 lpm, while their size relative to each other would establish the O₂% in the output mixture. Other total flow rates could be used and this is not a limitation of the invention. Also, it is noted that the pneumatic resistance 330 shown in the nozzle bypass line of FIGS. 5A-5C, could be removed and its function incorporated into the sizing of the nozzle bypass hole of the flow diverter. Another embodiment for the flow splitter units would utilize a sliding element as shown in FIG. 10. In this embodiment, the diverter slides a series of holes across the connection ports. Consequently the control of O₂% is done in a horizontal “pull” fashion rather than the rotational fashion of FIG. 6. Other embodiments exist, with the novel application of these designs to pneumatically control both total flow emerging from the venturi and relative split between the nozzle flow and nozzle bypass. It can also be observed that the transition between one O₂% setpoint to the next could be continuous in nature. Examining the cut-away of FIG. 6, the discrete holes that form the pneumatic resistances for each of the nozzle and bypass connections, could be continuous grooves in the diverter. In this way, as the diverter rotated counter-clockwise, the groove opening for the nozzle diverter connection would gradually get smaller, while the groove opening on the nozzle connection would be getting larger. Note that the grooves associated with each connection would not necessarily be identical, so as to provide the proper total flow (e.g. 40 lpm).

FIGS. 6-12 illustrate exemplary implementations of a flow splitter of a venturi-based oxygen concentration adjustor according to the present disclosure. An important factor of the design of the flow splitter is simplicity, e.g., to facilitate a low cost of manufacture, and ease of operation, and/or enable use of the ventilator by a user without the sophisticated training necessary to operate modern ventilators. The flow splitter includes a main body that defines an inlet, two outlets, and a fluid passage connecting the inlet to the two outlets. The flow splitter includes a diverting member (or simply diverter) operatively positioned with respect to the fluid passage (e.g., within the fluid passage) to selectively cause at least a portion of the total flow entering the fluid passage to exit out of the first outlet, the second outlet, or a combination of the first and second outlets. For example, the diverter may be movably coupled to the main body such that a change in the position of the diverter relative to the main body effects a different output state of the flow splitter. An actuator is operatively coupled to the diverter to selectively position the diverter into one of a plurality of positions, each of which effects a different output state of the flow splitter (e.g., a different split of the input flow). The actuator may be configured to position the diverter into one of a plurality of predetermined discrete positions, and may include a detent mechanism associated therewith to resist unintentional repositioning of the flow diverter out of the selected setting and/or provide tactile feedback when the flow splitter sets (e.g., clicks) into a predetermined setting. Alternatively, the diverter may be positionable by the actuator in a continuous, rather than discrete, manner to any setting between a minimum and a maximum setting thereof. To position the diverter into a different setting or position, the actuator may cause the diverter to rotate, translate, or a combination thereof, relative to the main body of the flow splitter. Repositioning of the diverter relative to the main body varies the restriction of flow out of each of the outlets thereby adjusting the relative amount of flow that exits the flow splitter through each of the outlets.

FIGS. 6A and 6B show an isometric view and a cutaway view, respectively, of a flow splitter 400 according to some examples of the present disclosure. The flow splitter 400 includes a main body 401, shown here as a hollow cylindrical (i.e. tubular) housing 410, which defines an inlet 402, a first outlet 404 and a second outlet 406. The main body 401 defines a fluid passage 405 (e.g., the central passage of the tubular housing 410) that fluidly couples the inlet 402 to both the first and second outlets 404 and 406, respectively. In use, the inlet 402 is fluidly connected to the O₂ supply, such that of flow of oxygen can be provided into the interior of the main body 401. In this embodiment, the first and second outlets 404 and 406, respectively, are shown on a diametrically opposite side of the housing 410 from the inlet 402. However, in other embodiments, the inlet 402, and outlets 404 and 406 may be differently arranged such as the outlet being positioned on a top side or a bottom side of the housing 410, or by placing one of the outlets on different sides of the housing (e.g., one on the top side and the other on the bottom side). One of the outlets (e.g., first outlet 402) functions as the venturi outlet and may thus be connected to the fluid line connecting the flow splitter to the inlet of the venturi device for delivering a flow of oxygen to the venturi nozzle. The other outlet (e.g., second outlet 406) functions as the bypass outlet, and is connected to a fluid line that bypasses the venturi nozzle. As such any flow directed to the first outlet is a flow that will facilitate entrainment, while flow directed to the bypass outlet modifies the composition of the gases (ambient air and bypass O₂) that are ultimately entrained through the diffuser 312 of the venturi device.

The flow splitter 400 includes a flow diverter 403 movably coupled to the main body 401. As such, the flow diverter 403 is configured to control the amount of flow that is permitted out of the first outlet 402 and the second outlet 404. In the illustrated example, the flow diverter 403 is implemented by a cylindrical or tubular insert 420 rotatably received in the tubular housing 410 and having an outer diameter sized for a clearance fit with the inner diameter of the tubular housing 410 such that the insert 420 may substantially freely rotatable within the housing 410. The flow splitter 410 may have be a relatively compact form factor, for example having a length of about 30 cm and a width (e.g., outer diameter of the main body 410) of about 9 cm. These dimensions are, of course, provided as a non-limiting example and it will be understood that the flow splitter may have different dimensions.

The tubular insert 420 is implemented by a hollow, substantially cylindrical body that defines a central passage 407, the axial ends of which are enclosed by a first wall 409 and a second wall 411 opposing or facing the first wall 409. It will be understood that the term axial refers to a direction extending generally along or parallel to the axis of the generally tubular flow splitter, or in the case of a non-cylindrical configuration, the longitudinal direction of the device. The term radial or diametric refers to directions transverse to the axial direction, i.e., directions generally perpendicular to the axial direction. The tubular insert 420 terminates at an actuation end (or simply actuator) 408. The actuator 408 may include a handle or knob provided in a location (e.g., external to the housing 410) accessible to the user such that the user can manipulate the handle or knob thereby changing the relative position of the tubular insert 420 with respect to the housing 410. In some examples, the actuation end 408 is integrally formed with the flow diverter 403, as is the case in the present example. In other embodiments, the two may be separately formed and fixedly joined such that the movement of the actuator 408 is synchronously transmitted to the diverter 403 to cause it to move relative to the main body 401. The handle or knob, when provided, may be separately formed and operatively coupled to the actuation end of the flow diverter 403 (e.g., via a keyed interface or by fixedly joining the two) to similarly effect the transmission of user force, in this case rotational force, to the flow diverter 403. The flow splitter may include a seal 426 that substantially seals the interior of the flow splitter to substantially prevent gases from escaping through the upper opening of the cylindrical housing 410, although a perfect seal is not necessary for a proper operation of the device. In this example, the seal 426 is implemented as an O-ring received in an annular groove in the integral body that forms the tubular insert and actuator.

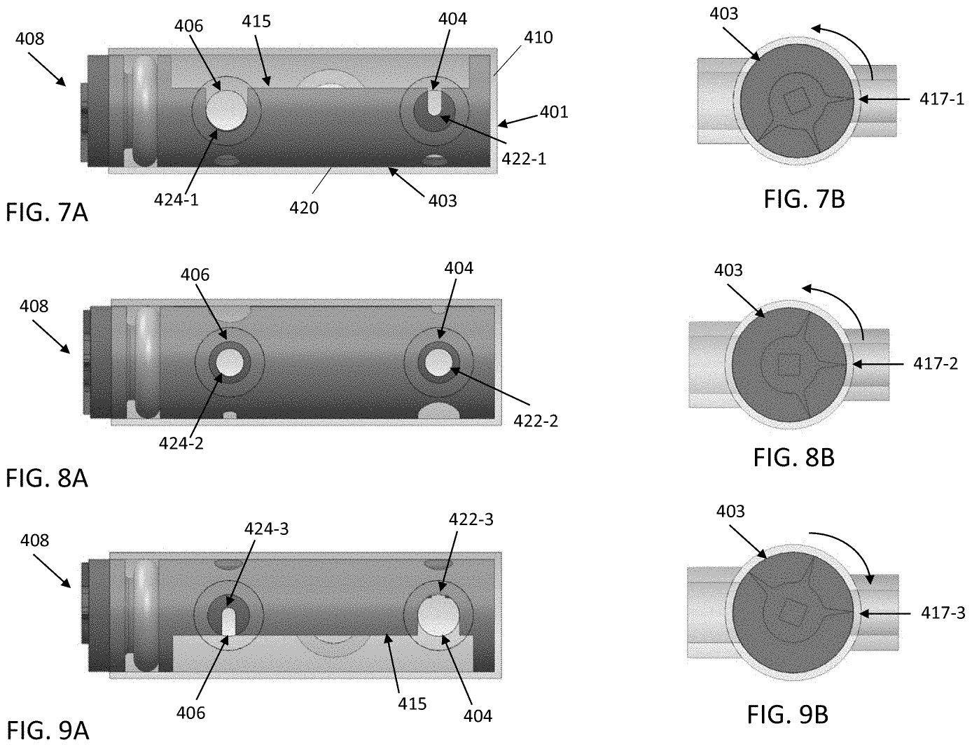

A cylindrical side wall 413 connects the opposing first and second walls 409 and 411. A portion of the cylindrical side wall 413 is cutaway forming a cutout 415 (see FIGS. 7A and 9A), which is aligned with the inlet 402 at all time (e.g., in all operable positions of the diverter 401, to allow the supply flow to enter the central passage 407. Two sets of openings, each opening of a given set having a different size, are formed on the remaining portion of the cylindrical side wall 413 at a longitudinal location of the side wall 413 in alignment with the respective outlet. The openings of the first set 422 are radially spaced at a first longitudinal location of the side wall 413 that enables each opening to substantially coaxially align with the aperture of the first outlet 404. Similarly, the openings of the second set 424 are radially spaced at a second longitudinal location of the side wall 413 that enables each opening to substantially coaxially align with the aperture of the second outlet 406.

In this specific example, because the flow splitter is configured to have three discrete settings 417, the first set of openings 422 and the second set of openings 424 each include three differently sized openings. The first set of openings 422 includes a first opening 422-1, which is the smallest opening in the set 422 and is substantially axially aligned with the first opening 424-1, which is the largest opening of the set 424. As such, when the diverter 403 is provided in its first setting 417-1, as shown in FIGS. 7A and 7B, in which the first (smallest) opening 422-1 of the first set 422 is aligned with the first (venturi) outlet 404 and the first (largest) opening 424-1 of the second set 424 aligns with the second (bypass) outlet 406, the relatively largest amount of oxygen flow is diverted to the venturi bypass resulting in an output flow containing the highest oxygen concentration. A second opening 422-2 of the first set 422 and a second opening 424-2 of the second set 424 are substantially axially aligned, and are substantially the same size (e.g., same diameter). The diverter 403, thus, splits the flow of supply oxygen, in its second setting 417-5 shown in FIGS. 8A and 8B, about evenly between the two outlets, resulting in an output flow of lower oxygen concentration as compared to the first setting. A third opening 422-3 of the first set 422, which is the largest sized opening of the first set is substantially axially aligned with the third opening 424-3, having the smallest size of all of the openings of the second set 424. As such, when the diverter 403 is provided in its third setting 417-3, as shown in FIGS. 9A and 9B, in which the third (largest) opening 422-3 of the first set 422 is aligned with the first (venturi) outlet 404 and the third (smallest) opening 424-3 of the second set 424 aligns with the second (bypass) outlet 406, the relatively smallest amount of oxygen flow is diverted to the venturi bypass resulting in an output flow containing the lowest oxygen concentration as compared to the other two settings. It will be understood that the different pairs of openings (e.g., first openings 422-1 and 424-1, second openings 422-2 and 424-2) are substantially axially aligned in this example because the outlets 404 and 406 are also aligned along the wall of the cylindrical housing 410. However, in other examples in which the outlets 404 and 406 are differently arranged, the pairs of the openings may also be differently arranged to achieve an equivalent function as described herein. The flow diverter 403 may be rotated to each of the three rotational positions corresponding to the three different settings by a user manipulating the actuator 408. In other examples, the repositioning of the flow diverter 403 relative to the main body 401 for changing the setting, and thus oxygen concentration of the output flow, may be achieved through electronic control (e.g., via a solenoid) rather than by manual force. In one specific embodiment, the size of largest opening in each set (e.g., first opening 424-1 and third opening 424-3) is about 3× the size of the smallest opening in each set (e.g., third opening 424-3 and first opening 424-1) and is about 2× the size of the middle opening in each set (e.g., second openings 424-2 and 424-2). In one specific example the size of the largest openings is about 3 cm, the size of the middle openings is about 2 cm and the size of the smallest openings is about 1 cm. It will be understood, however, that the sides of the openings may be different in other embodiments depending on the total flow rate or volume desired at the output.

In the preceding example, the flow diverter is rotatable relative to the main body to reconfigured to flow splitter between settings and thus change the oxygen concentration in the inspiratory flow. In other examples, the flow diverter may be differently movably associated with the main body. For example, the diverter may translate or slide relative to an axial direction of the main body for changing the flow splitter setting. FIG. 10 shows a cross-sectional simplified illustration of a flow splitter 500 according to further embodiments herein. The flow splitter 500 of FIG. 10 includes a main body (also referred to as housing) 501 that defines an inlet 502, a first outlet 504, a second outlet 506, and a fluid passage 507 that fluidly connects the inlet 502 to the first and second outlets 504 and 506, respectively. In the absence of the diverting member 503, the flow entering the fluid passage 505 through inlet 502 would be permitted to exit, substantially unobstructed, via either of the outlets 504 and 506. A flow control member (also referred to as diverter or insert) 503 is slidably received in the passage 505 and is configured to selectively restrict and/or prevent flow through the first outlet 504, the second outlet 506, or both. To that end, the diverter 503 is movably coupled within the fluid passage 505 and configured to selectively occlude at least a portion of the first outlet 504, the second outlet 506, or both. An actuator 508 may be fixed to the diverter 503 and extend to a location accessible to the user (e.g., a location exterior to the main body 501) to enable a user to manipulate the actuator 508 to move the diverter relative to the main body 501.

The diverter 503 may be implemented as a hollow (e.g. monolithic) body that defines an inlet opening 515, one or a plurality of first outlet openings 522, one or a plurality of second outlet openings 524, and a central passage 507 that connects the inlet opening 515 to each of the first outlet openings 522 and second outlet openings 524 such that the fluid (e.g., oxygen) can be freely communicated from the inlet opening 515 to the outlet openings 522 and 524. In the example in FIG. 10, the diverter 503 includes a plurality of first outlet openings 522, each of which is differently sized. In the present example, in each set, the opening closest to the actuator 508 is largest in size, the size of each adjacent opening in a given set decreasing in from the actuation end toward the opposite end of the diverter 503. This sizing order may be reversed in other examples, or a different sizing order may be used, such as by longitudinally ordering the openings from small to large in one set and from large to small in the other set, as long as a mismatched alignment is achieved whereby when a greater amount of flow if allowed through one of the first or second outlets 504 and 506, a lesser amount of flow is allowed through the other one of the first and second outlets 504 and 506. This can be achieved with a plurality of discrete, spaced apart, outlet openings 522 and 524 of different sizes included in each set or with a single opening 522 and 524 suitably positioned to provide the mismatched outflow through the respective one of the outlets 504 and 506, as shown in the example in FIG. 10. The diverter 503 may be slidable to one of four discrete positions, in each of which a different combination of the first and second openings 522 and 524 are aligned with the respective one of the first and second outlets 504 and 506. Alternatively, the diverter 503 may be slidable or adjustable continuously to any intermediate position between the outbound range of motion of the slidable diverter 503. One such position is shown in FIG. 10 in which the aperture of the first outlet 504 is aligned with, and thus permits flow through, the first three discrete openings 522, while at the same time the second outlet 506 is aligned with, and thus permits flow through, only the last (here, the fourth) discrete opening 524. While each of the openings in each set is shown to have a different size, in other examples, each opening may have the same size and the relative amount of flow permitted out of a given one of the two outlets may depend upon the total number of openings 522 or 524 that are aligned with the respective outlet 504 and 506.

FIG. 11 shows another example of a flow splitter 500′ which is similarly configured with a slidable diverter 503 like the flow splitter 500. The flow splitter 500′ of FIG. 11 and the flow splitter 500 of FIG. 10 share many similar components and operate similarly with a key difference being that instead of having discrete outlet openings as in the example in FIG. 10, the diverter 503 in this example has a single first outlet opening 522 selectively alignable to the first outlet 504 and a single second outlet opening 524 selectively alignable to the first outlet 506. The diverter 503 may translate or slide along the direction 509 to selectively vary the amount of occlusion of each of the outlets 504 and 506. Like the flow splitter 500 of FIG. 10, the inlet opening 515 facing the side of the housing that includes the inlet 502 is sufficiently large such that the inlet 502 is substantially unobstructed by the diverter 503 in any adjustment position (or setting) of the diverter 503. In use, when the diverter 503 is slidingly provided to a first, minimum oxygen concentration setting, the first outlet opening 522 may be substantially fully aligned with the aperture of the first outlet 504 such that the first outlet 504 is substantially unobstructed, while the second outlet opening 524 is fully misaligned with the second outlet 506 such that the second outlet 506 is substantially fully obstructed by the diverter 503. As such, in this first setting, substantially all of the flow entering the flow splitter through inlet 502 is directed to the first outlet 504. In another setting, corresponding to a maximum oxygen concentration setting, the first outlet opening 522 is substantially fully misaligned with the aperture of the first outlet 504 such that the first outlet 504 is substantially fully obstructed, while the second outlet opening 524 is substantially fully aligned with the second outlet 506 such that the second outlet 506 is substantially obstructed, thereby diverting or directing all of the flow to the second outlet 506. The diverter 503 may be continuously adjustable to any intermediate position between these two settings to vary the amount of flow directed to each of the first and second outlets 504 and 506, and thus vary the resulting oxygen concentration in the output flow. It may be understood that first outlet opening 522 and second outlet opening 524 may be achieved as non-linear through cut grooves in diverter 503, and need not be symmetrical or identical in shape or design. In other embodiments, even in the case of using a single outlet opening for each outlet, the flow diverter 503 may be detented or adjustable to discrete intermediate positions between the minimum and maximum oxygen concentration settings.

The flow control member or diverter 503 in the examples in FIGS. 10 and 11 is movably coupled such that it translates along a direction generally transverse to the flow. The flow control member 503 may be implemented by a tubular structure, for example in cases in which the main body 501 is also a tubular structure with a tubular central passage. The flow control member may slide within a tubular central passage of the housing. As such, the housing and insert may have generally circular transverse cross sections. In other embodiments of the slidably adjustable flow splitter, the diverter and/or housing may have a different, non-circular cross section. Typically, the outer shape of the diverter corresponds in shape to the shape of the central passage of the main body to enable substantially unobstructed movement (e.g., sliding) of the diverter therein. Also, while exemplary flow splitters are described as configured for manual operation, it will be understood that in some embodiments the flow splitter may be electronically controlled, such as by a solenoid operating, for example, responsive to controller 100, to change the setting thereof.

As previously noted, a flow splitter according to the present disclosure may be adjustable to one of a plurality of discrete flow splitter settings, each associated with a corresponding discrete oxygen level concentration. The flow diverter may thus be associated with a detent mechanism, which resists the relative movement (e.g., rotational or translational) of the flow diverter from a discrete setting. In some instances the detent mechanism may also be configured to urge the flow diverter to a position corresponding to a discrete setting, when the flow diverter is moved to a position in between settings. An example of a detent mechanism is shown in FIG. 12 as a ball and spring mechanism 600. The ball and spring mechanism 600 may be attached to an anchor structure 602, such as the main body (e.g., housing 410). The ball and spring mechanism 600 may engage one of a plurality of notches 605, provided in the moving structure 604 (e.g., the first wall or the second wall of the tubular insert). The ball 601 of the mechanism 600 may be urged away from the anchor structure 602 by spring 603 such that the ball 601 is received in a notch 605 when the diverter is slidably or rotationally aligned with the main body (e.g., housing 410) in a position corresponding a discrete setting. The spring's stiffness may be sufficiently high to prevent accidental or unintentional repositioning (e.g., rotation or translation) of the diverter (e.g., as may be caused by movement of the ventilator core) but sufficiently low to ensure that the diverter can be repositioned responsive to manual force of a typical adult operator. While an example ball and spring detent mechanism is shown for illustration purposes only, any other suitable detent mechanism may be used for resisting the relative movement of the diverter out of the detented position and in some cases, to optionally urge the diverter into a particular setting.

The foregoing description has broad application. The discussion of any embodiment is meant only to be explanatory and is not intended to suggest that the scope of the disclosure, including the claims, is limited to these examples. In other words, while illustrative embodiments of the disclosure have been described in detail herein, the inventive concepts may be otherwise variously embodied and employed, and the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more aspects, embodiments, or configurations for the purpose of streamlining the disclosure. However, various features of the certain aspects, embodiments, or configurations of the disclosure may be combined in alternate aspects, embodiments, or configurations. Moreover, the following claims are hereby incorporated into this Detailed Description by this reference, with each claim standing on its own as a separate embodiment of the present disclosure.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative sizes reflected in the drawings attached hereto may vary.

What is claimed is:

1. A modular ventilator comprising:
a ventilation core comprising an inlet port for coupling an internal pneumatic circuit of the ventilation core to an external source of pressurized O₂, wherein the internal pneumatic circuit comprises an O₂ concentration regulator, which includes a venturi device and a flow splitter, the flow splitter configured to adjustably control a relative amount of the pressurized O₂ that is provided to a nozzle of the venturi device and to a fluid line that bypasses the nozzle; and
a service module removably coupled to the ventilation core, the service module is configured to receive data from the electronic controller of the ventilation core for monitoring and displaying additional information and/or alarms to any information and/or alarms generated by the ventilation core; and wherein:
the ventilation core includes or is connectable to a power source independent of any power source of the service module such that the ventilation core is operable independent of the service module being connected thereto.

2. The modular ventilator of claim 1, wherein the ventilation core is a single fault system configured to generate an alarm only when one predetermined parameter fails to meet a safe operation criteria.

3. The modular ventilator of claim 2, wherein the one predetermined parameter is peak inspiratory pressure (PIP), and wherein the ventilation core is configured to generate an alarm only when the PIP exceeds a predetermined maximum PIP value.

4. The modular ventilator of any of the preceding claims, wherein the ventilation core is configured to transmit power to the service module when the service module is connected thereto.

5. The modular ventilator of any of the preceding claims, wherein the ventilation core comprises a rechargeable battery and the service module is configured to transmit power to the ventilation core for recharging the battery of the ventilation core when the service module is connected thereto.

6. The modular ventilator of any of the preceding claims, wherein the ventilation core comprises an electronic controller configured to control an inspiratory valve and an expiratory valve of the ventilation core but not the O₂ concentration regulator.

7. The modular ventilator of any of the preceding claims, wherein an O₂-air mixture output by the O₂ concentration regulator is varied by manually repositioning a moving component of the flow splitter.

8. The modular ventilator of any of the preceding claims, wherein the pneumatic circuit further comprises:
a pressure regulator configured to reduce the pressure of the pressurized O₂ to provide a pressure-regulated flow at its output;
an inspiratory valve which couples the pressure-regulated flow to the O₂ concentration regulator when the inspiratory valve is open to produce an inspiratory flow of O₂-air mixture; and
a pressure relief valve downstream of the O₂ concentration regulator and configured to open if the pressure of the inspiratory flow reaches a predetermined maximum pressure.

9. The modular ventilator of claim 8, wherein the ventilation core includes an inspiratory port and an expiratory port, both configured to be connected to an external flow sensor via tubing, and wherein the pressure relief valve opens responsive to measurements obtained by the external flow sensor.

10. The modular ventilator of claim 8, wherein the inspiratory valve is an electronically-controlled normally closed valve, the pneumatic circuit further comprising an expiratory valve which is an electronically-controlled normally open valve and is configured to close in synchrony with opening of the inspiratory valve.

11. A ventilation core of a modular ventilator, the ventilation core comprising:
an inlet configured to receive a supply flow of pressurized O₂;
a pressure regulator configured to reduce the pressure of the supply flow and output a reduced-pressure flow;
an inspiratory valve configured to receive the reduced-pressure flow, wherein the inspiratory valve comprises a normally closed valve configured to selectively shift to the open position to permit flow of the reduced-pressure flow to downstream components of the pneumatic circuit during an inspiration phase of a breathing cycle; and
an O₂-air mixture adjustor configured to receive the reduced-pressure flow and output an inspiratory flow having a selected O₂ concentration, wherein the O₂-air mixture adjustor comprises a venturi nozzle and a flow splitter upstream of the venturi nozzle, and wherein the flow splitter is configured to selectively divert at least a portion of the reduced-pressure flow to bypass the venturi nozzle based on a setting of the O₂-air mixture adjustor.

12. The ventilation core further comprising:
an inspiratory port configured to transmit the inspiratory flow out of the ventilation core for delivery to a subject;
an expiratory port configured to receive an expiratory flow from the subject for exhausting the expiratory flow through an outlet of the ventilation core; and
an expiratory valve coupled between the expiratory port and the outlet to selectively permit the exhausting of the expiratory flow through the outlet.

13. The ventilation core of claim 12, an adjustable valve between the expiratory port and the outlet, wherein the adjustable valve is configured to close to prevent flow therethrough if the pressure of the expiratory flow falls to a predetermined minimum value.

14. The ventilation core of claim 12, wherein the expiratory valve is a normally open valve configured to shift to a closed state in synchrony with opening of the inspiratory valve.

15. The ventilation core of claim 13, wherein the inspiratory and expiratory valves are electronically controlled valves, the ventilation core further comprising a controller communicatively coupled to the inspiratory and expiratory valves to transmit control signals for opening and closing of the inspiratory and expiratory valves.

16. The ventilation core of any of the preceding claims, wherein the flow splitter comprises:
an outer body defining an inlet configured to receive the reduced-pressure flow, a first outlet connected to the venturi nozzle and a second outlet connected to a fluid line bypassing the venturi nozzle, and a fluid passage connecting the inlet to the first and second outlets; and
a diverter comprising an inner body received within the fluid passage and movably relative to the fluid passage to selectively occlude, at least partially, the first and/or second outlets.

17. The ventilation core of claim 16, wherein the diverter comprises a tubular insert comprising a first set of holes, each having a different side, radially spaced at a first longitudinal location of the tubular insert corresponding to a location of the first outlet, and a second set of holes, each having a different size, radially spaced at a second longitudinal location of the tubular insert corresponding to a location of the second outlet, and wherein the tubular insert is rotatable within the fluid passage to selectively align different pairs of the holes, each pair including one hole of the first set of holes and one hole of the second set of holes, to vary the relative pneumatic resistance to flow out of first and second outlets.

18. The ventilation core of claim 16, wherein the diverter comprises an insert slidably coupled to the fluid passage and configured to translate along a longitudinal direction of the fluid passage to selectively occlude, at least partially, the first and/or second outlets.

19. An apparatus for reducing oxygen concentration of an O₂ supply flow of a ventilator, the apparatus comprising:
a venturi device comprising a nozzle having a constricted section;
a nozzle feed line connected upstream of the constricted section;
a bypass line connected downstream of the constricted section, wherein the bypass line is fluidly coupled, via a one way valve, to ambient air, the one way valve permitting flow only in a direction from the ambient air into the bypass line;
a flow splitter comprising:
an outer body defining a first outlet connected to the nozzle feedline and a second outlet connected to the bypass line; and
an inner body defining one or more first openings associated with the first outlet and one or more second openings associated with the second outlet, wherein the one or more first openings are configured to provide pneumatic resistance to flow out of the first outlet based on relative alignment therebetween and the one or more second openings are configured to provide pneumatic resistance to flow out of the second outlet based on a relative alignment therebetween, and wherein the inner body is movably coupled to the outer body to selectively vary the alignment of the one or more first openings and one or more second openings relative to the respective one of the first and second outlets thereby varying pneumatic resistance to flow out of each of the first and second outlets.

20. The apparatus of claim 19, wherein the outer body defines a cylindrical passage, and wherein the diverter comprises a tubular insert rotatably received within the cylindrical passage.

21. The apparatus of claim 20, wherein the tubular insert comprises a first set of holes, each having a different side, radially distributed at a first longitudinal location of the tubular insert corresponding to a location of the first outlet, and a second set of holes, each having a different size, radially distributed at a second longitudinal location of the tubular insert corresponding to a location of the second outlet, such that rotation of the tubular insert within the cylindrical passage selectively aligns a different pair including one hole of the first set of holes and one hole of the second set of holes, with the first and second outlets.

22. A ventilator according to any of the examples herein.

23. A ventilation core of a modular ventilator according to any of the examples herein.

24. An apparatus for adjusting oxygen concentration in inspiratory flow provided by a ventilator according to any of the examples herein.

25. A method of mechanically ventilating a subject according to any examples herein.

26. A method of electronically controlling a pneumatic circuit of a ventilator according to any of the examples herein.

27. A ventilator controller according to any of the examples herein.

28. A pneumatic circuit of a ventilator according to any of the examples herein.
